(19) United States
(12) Patent Application Publication
(10) Pub. No.: US 2026/0207100 A1
(43) Pub. Date: Jul. 23, 2026

(54) BLOOD VESSEL VISUALIZATION DEVICE, BLOOD VESSEL VISUALIZATION SYSTEM, AND BLOOD VESSEL VISUALIZATION METHOD

(71) Applicants: TERUMO KABUSHIKI KAISHA, Tokyo (JP); INSTITUTE OF SCIENCE TOKYO, Tokyo (JP)

(72) Inventors: Shota TSUMAKI, Nakakoma-gun (JP); Yoshihiro WATANABE, Tokyo (JP)

(73) Assignees: TERUMO KABUSHIKI KAISHA, Tokyo (JP); INSTITUTE OF SCIENCE TOKYO, Tokyo (JP)

(21) Appl. No.: 19/572,174

(22) Filed: Mar. 19, 2026

Related U.S. Application Data
(63) Continuation of application No. PCT/JP2024/029103, filed on Aug. 15, 2024.

(30) Foreign Application Priority Data
Sep. 25, 2023 (JP) 2023-161607

(51) Int. Cl. A61B 5/15; A61B 5/00

(52) U.S. Cl. CPC A61B 5/150053; A61B 5/0077

(57) ABSTRACT

A non-transitory computer-readable medium containing a computer program that, when executed, causes a computer to execute processing including: detecting blood vessel information of a blood vessel included in a predetermined site of a subject based on an image obtained by imaging the predetermined site; generating a projection image of the blood vessel based on the detected blood vessel information; and while causing at least one projector to project the generated projection image, causing the at least one projector to project a virtual operation unit for operating the projection image onto a region excluding a blood vessel region included in the projection image.

### CROSS-REFERENCE TO RELATED APPLICATIONS

This application is a bypass continuation of PCT Application No. PCT/JP2024/029103, filed on Aug. 15, 2024, which claims priority to Japanese Patent Application No. 2023-161607, filed on Sep. 25, 2023. The entire contents of these application are hereby incorporated by reference herein.

### BACKGROUND

The present disclosure relates to a computer program, a blood vessel visualization device, a blood vessel visualization system, and a blood vessel visualization method.

A medical worker performs puncture (venipuncture) in various situations such as blood sampling, drip infusion, and dialysis, but it may be difficult to find a blood vessel suitable for puncture in a case where a blood vessel of a person to be punctured is thin.

Japanese Patent Publication No. 2004-237051 A (“Patent Literature 1”) discloses a blood vessel visualization device capable of projecting a blood vessel image on a monitor by irradiating an inner side of a human forearm with near-infrared light, and puncturing a blood vessel with an injection needle of a syringe by targeting the blood vessel suitable for puncturing.

### SUMMARY

However, in the blood vessel visualization device as disclosed in Patent Literature 1, it is necessary to operate a device button or the like at a position away from the puncture site. When a medical worker performs puncturing, it is difficult to operate the device button because both hands are used. In addition, when the device button is operated during puncture, there is also concern about infection via the device button.

The present disclosure has been made in view of such circumstances, and an object of the present disclosure is to provide a computer program, a blood vessel visualization device, a blood vessel visualization system, and a blood vessel visualization method capable of supporting easy puncturing without operating a device button.

(1) A non-transitory computer-readable medium containing a computer program, when executed, causes a computer to execute processing comprising: detecting blood vessel information of a blood vessel included in a predetermined site of a subject based on an image obtained by imaging the predetermined site; generating a projection image of the blood vessel based on the detected blood vessel information; and while causing at least one projector to project the generated projection image, causing the at least one projector to project a virtual operation unit for operating the projection image onto a region excluding a blood vessel region included in the projection image.

Here, according to embodiments of the present disclosure,
(2) the computer program of the above (1) further causes the computer to execute processing of detecting a projectable region of the virtual operation unit, and causing the one or more projectors to project the virtual operation unit onto a region that is the detected projectable region and excludes the blood vessel region.

(3) The computer program of the above (1) or (2) further causes the computer to execute processing of detecting an operation on the virtual operation unit; and changing display of the projection image in accordance with the detected operation.

(4) The computer program according to any one of the above (1) to (3) further causes the computer to execute processing of specifying a projection recommendation region of the virtual operation unit, and causing the one or more projectors to project the virtual operation unit onto the specified projection recommendation region.

(5) The computer program of the above (4) further causes the computer to execute processing of detecting a movement range of an arm including a finger of a puncture operator at the predetermined site, and specifying the projection recommendation region by excluding the detected movement range.

(6) In the computer program of the above (5), the projection recommendation region is a region excluding the movement range and includes a region close to the puncture operator.

(7) The computer program according to any one of the above (1) to (6) further causes the computer to execute processing of calculating blood vessel parameters including at least one of a length, a thickness, a depth, a straightness, and a traveling direction of the blood vessel on the basis of the detected blood vessel information, specifying a puncture optimal blood vessel region among blood vessel regions included in the projection image on the basis of the calculated blood vessel parameters, and causing the one or more projectors to project the virtual operation unit onto a region excluding the puncture optimal blood vessel region.

(8) The computer program according to any one of the above (1) to (7) further causes the computer to execute processing of calculating blood vessel parameters including at least one of a length, a thickness, a depth, a straightness, and a traveling direction of the blood vessel on the basis of detected blood vessel information, calculating a projection avoidance priority of a blood vessel region included in the projection image by performing weighting with the calculated blood vessel parameters, and selecting a projection region onto which the virtual operation unit is to be projected on the basis of the calculated priority.

(9) The computer program according to any one of the above (1) to (8) further causes the computer to execute processing of detecting a position of a blood vessel access device that punctures the predetermined site on the basis of the image obtained by imaging the predetermined site, and causing the one or more projectors to project the virtual operation unit onto a region other than the detected position.

(10) The computer program according to any one of (1) to (9) further causes the computer to execute processing of selecting an operation target object of the virtual operation unit according to priorities of a plurality of operation target objects included in the virtual operation unit, and causing the one or more projectors to project the selected operation target object.

(11) A blood vessel visualization device comprises: a control unit configured to: detect blood vessel information of a blood vessel included in a predetermined site of a subject based on an image obtained by imaging the predetermined site, generate a projection image of the blood vessel based on the detected blood vessel information, and while causing one or more projectors to project the generated projection image, cause the one or more projectors to project a virtual operation unit for operating the projection image onto a region excluding a blood vessel region included in the projection image.

(12) A blood vessel visualization system according to the present disclosure includes the above blood vessel visualization device and a blood vessel access device that punctures a predetermined site of a subject.

(13) A blood vessel visualization method comprises: detecting blood vessel information of a blood vessel included in a predetermined site of a subject based on an image obtained by imaging the predetermined site; generating a projection image of the blood vessel based on the detected blood vessel information; and while projecting the generated projection image, projecting a virtual operation unit for operating the projection image onto a region excluding a blood vessel region included in the projection image.

According to the present disclosure, it is possible to support easy puncturing without operating a device button.

### BRIEF DESCRIPTION OF THE DRAWINGS

FIG. 1 is a diagram illustrating an example of a configuration of a blood vessel visualization device of the present embodiment.

FIG. 2 is a diagram schematically illustrating a first example of a method of mounting the blood vessel visualization device.

FIG. 3 is a diagram schematically illustrating a second example of a method of mounting the blood vessel visualization device.

FIG. 4 is a diagram illustrating an example of blood vessel information (blood vessel image).

FIG. 5 is a diagram illustrating an example of blood vessel parameters.

FIG. 6 is a diagram illustrating an example of a projection image of a blood vessel.

FIG. 7 is a diagram illustrating an example of a projection region of a virtual operation unit.

FIG. 8 is a diagram illustrating an example of a method of calculating a puncture blood vessel priority and a projection avoidance blood vessel priority.

FIG. 9A is a diagram illustrating an example of a virtual operation unit.

FIG. 9B is a diagram illustrating an example of a virtual operation unit.

FIG. 9C is a diagram illustrating an example of a virtual operation unit.

FIG. 10 is a diagram illustrating an example of a virtual operation unit during projection.

FIG. 11 is a diagram illustrating an example of an indirect operation on a virtual operation unit.

FIG. 12 is a diagram illustrating an example of a configuration of a blood vessel visualization system including a blood vessel access device.

FIG. 13 is a view illustrating an example of a processing procedure performed by the blood vessel visualization device.

### DETAILED DESCRIPTION

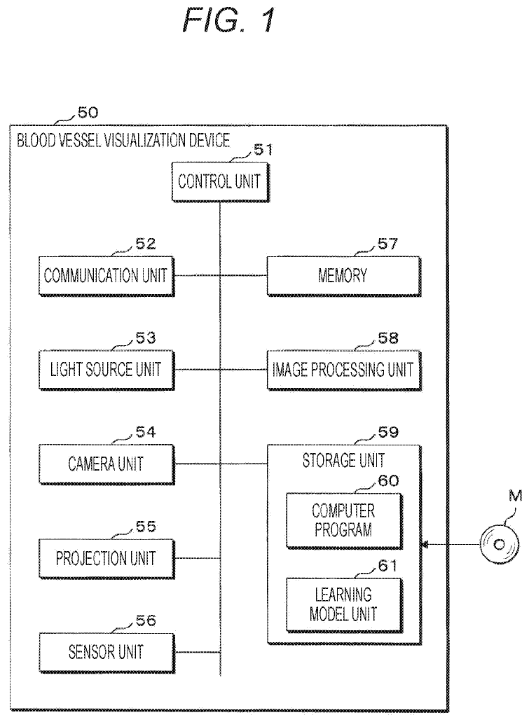

Hereinafter, embodiments of the present disclosure will be described. FIG. 1 is a diagram illustrating an example of a configuration of a blood vessel visualization device 50 according to the present embodiment. The blood vessel visualization device 50 includes a control unit 51 that controls the entire device, a communication unit 52, a light source unit 53, a camera unit 54, a projection unit 55, a sensor unit 56, a memory 57, an image processing unit 58, and a storage unit 59.

The control unit 51 is configured by incorporating a required number of central processing units (CPUs), micro-processing units (MPUs), graphics processing units (GPUs), and the like. Furthermore, the control unit 51 may be configured by combining a digital signal processor (DSP), a field-programmable gate array (FPGA), and the like.

The communication unit 52 includes, for example, a communication module and may have a communication function (either wireless communication or wired communication may be used, or both may be used) with an external device (not illustrated).

The light source unit 53 includes, for example, one or a plurality of LED light sources that emit near infrared light of 700 to 2500 nm. The light source unit 53 includes, for example, one or a plurality of LED light sources that emit visible light of 380 to 770 nm. The light amount of the light source unit 53 can be adjusted under the control of the control unit 51. The light source unit 53 irradiates a predetermined site (a site to be punctured) of a subject (puncture subject) with the above-described light.

The camera unit 54 may capture, for example, near-infrared light of 700 to 2500 nm. Furthermore, the camera unit 54 may capture visible light of 380~770 nm, for example. Furthermore, the camera unit 54 may capture far-infrared light of 2500 nm or more, for example. The camera unit 54 may be configured as an identical body by integrating cameras capable of capturing each of near-infrared light, visible light, and far-infrared light, or may be configured as a separate body. In the case of the identical body, near-infrared light, visible light, and far-infrared light may be switched by using a filter or the like. In addition, in the case of the separate body, it is preferable that each camera can perform imaging coaxially by using a half mirror or the like.

In order to image a predetermined site of a subject, the camera unit 54 is configured to image the predetermined site from a position separated from the predetermined site by an appropriate length. In this case, the light source unit 53 may be disposed in the vicinity of the camera unit 54 on the same side as the camera unit 54 with respect to the predetermined site, or may be disposed on the side opposite to the camera unit 54 with respect to the predetermined site (that is, the camera unit 54 and the light source unit 53 are disposed with the predetermined site interposed therebetween), or the light source unit 53 may be disposed on both sides of the predetermined site with the predetermined site interposed therebetween.

The projection unit 55 includes a projector capable of projecting a projection image of a blood vessel and a virtual operation unit (digital object) for operating the projection image onto a required location such as a human skin or a desk surface. Note that the projector that projects the projection image and the projector that projects the virtual operation unit may be the same or separate. Details of the projection image and the virtual operation unit will be described later.

Furthermore, the camera unit 54 captures an image of an operation of a puncture operator (medical worker) with respect to the virtual operation unit. The control unit 51 can specify the content of the operation of the puncture operator on the basis of the image captured by the camera unit 54. For example, it is possible to specify an operation item of the virtual operation unit on the basis of movement of a finger or a hand of the puncture operator, a relative position with respect to an operation target object in the virtual operation unit, and the like. In addition, a learning model (one learning model of the learning model unit 61) trained to output assumed operation content in a case where an image of a finger or a hand including a virtual operation unit is input may be used.

The sensor unit 56 detects, for example, movement of a finger or a hand of a puncture operator such as a medical worker, and detects an operation on the virtual operation unit. The sensor unit 56 includes at least one of, for example, an acoustic sensor, a vibration sensor, a depth sensor, an acceleration sensor, and a potential sensor.

The acoustic sensor or the vibration sensor can detect vibration based on the motion of the arm or the palm (for example, vibration due to contact and vibration at the time of flicking), and determine a position where the puncture operator has flicked or a flicking manner (that is, differentiation of the operation of the puncture operator) on the basis of a shape or a strength of the detected vibration waveform.

By using a ring with the depth sensor worn on a wrist of a subject, the depth sensor irradiates a target site (for example, a finger or an arm) with infrared light, measures a time from the time of the irradiation until the reflected light returns, and can determine a position (coordinates) of the target site (a finger or the like) or a change in the target site (an arm or the like) on the basis of the measured time.

For example, by using a ring with the acceleration sensor worn on the puncture operator, the acceleration sensor detects the acceleration of the movement of the finger of the puncture operator, calculates the movement of the finger (the magnitude of movement, the number of movements, a direction of movement, and the like) on the basis of the detected acceleration, and can determine the movement of the finger.

For example, by using a ring with a potential sensor worn on the puncture operator, the potential sensor detects a potential difference at the time of skin contact, and can determine the presence or absence of contact, a position of contact, and the like on the basis of the detected potential difference.

The memory 57 may include a semiconductor memory, such as a static random access memory (SRAM), a dynamic random access memory (DRAM), or a flash memory.

The image processing unit 58 detects a blood vessel image of a blood vessel included in a predetermined site on the basis of an image obtained by imaging the predetermined site of the subject with the camera unit 54, and detects blood vessel information of the blood vessel. Furthermore, the image processing unit 58 detects a projectable region of the virtual operation unit on the basis of an image obtained by imaging a predetermined site of the subject with the camera unit 54. In addition, the image processing unit 58 detects a direct or indirect operation on the virtual operation unit by the puncture operator. The direct operation on the virtual operation unit is an operation of directly touching the projected virtual operation unit, and the indirect operation is an operation of operating an operation target different from the virtual operation unit to indirectly operate the virtual operation unit.

The storage unit 59 may be configured by, for example, a hard disk or a semiconductor memory, and may store a computer program (program product) 60, a learning model unit 61, and necessary information.

The computer program 60 is a computer program (application) that operates in the blood vessel visualization device 50, is loaded to the memory 57, and is executed by the control unit 51. That is, the processing executed by the control unit 51 corresponds to processing executed in accordance with the computer program 60. The computer program 60 may be installed by reading the computer program 60 recorded on a recording medium M (for example, an optically readable disc storage medium such as a CD-ROM) with a recording medium reading unit (not illustrated) and storing the computer program 60 in the storage unit 59, or by downloading the computer program 60 from an external device via the communication unit 52. Details of the learning model unit 61 will be described later.

Next, a method of mounting the blood vessel visualization device 50 will be described.

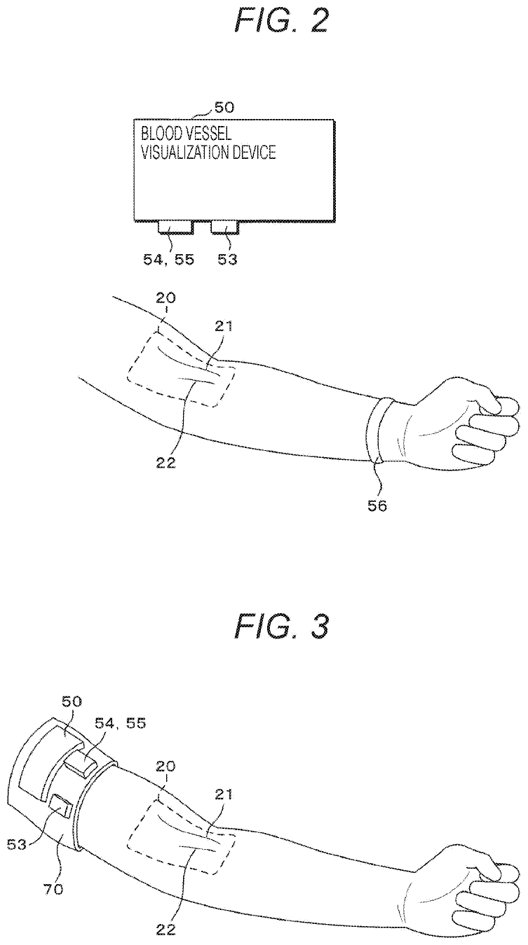

FIG. 2 is a diagram schematically illustrating a first example of a method of mounting the blood vessel visualization device 50. Hereinafter, an arm (forearm) will be described as an example of a predetermined site of the subject, but the predetermined site is not limited to the arm, and may be another site to be punctured in the case of blood sampling, drip infusion, dialysis, or the like. The blood vessel visualization device 50 is fixed to a support such as a stand to be disposed at a position away from the arm of the subject by an appropriate distance. Note that the blood vessel visualization device 50 may be movably attached to the support so that the light source unit 53, the camera unit 54, and the projection unit 55 are directed in an optimum direction. The sensor unit 56 is, for example, a depth sensor, but in the case of another sensor, the sensor unit 56 may be provided in the blood vessel visualization device 50 or may be mounted on the finger of the puncture operator or the like. Furthermore, in a case where the movement of the finger or the hand of the puncture operator is detected on the basis of the image captured by the camera unit 54, the sensor unit 56 need not be provided.

A projection image 20 of a blood vessel is projected onto the arm of the subject by the projection unit 55. In the example in FIG. 2, two blood vessel regions 21 and 22 are displayed in the projection image 20.

FIG. 3 is a diagram schematically illustrating a second example of a method of mounting the blood vessel visualization device 50. In the second example illustrated in FIG. 3, a mounting band 70 is mounted on the upper arm of the subject, and the blood vessel visualization device 50 is mounted on the mounting band 70. The light source unit 53, the camera unit 54, and the projection unit 55 are attached to appropriate positions of the mounting band 70 to be directed in an optimum direction. As in the case of the first example, the projection image 20 of a blood vessel is projected onto the arm of the subject by the projection unit 55, and the two blood vessel regions 21 and 22 are displayed in the projection image 20.

Next, a method of detecting blood vessel information of the arm of the subject on the basis of an image obtained by imaging the arm of the subject with the camera unit 54 will be described.

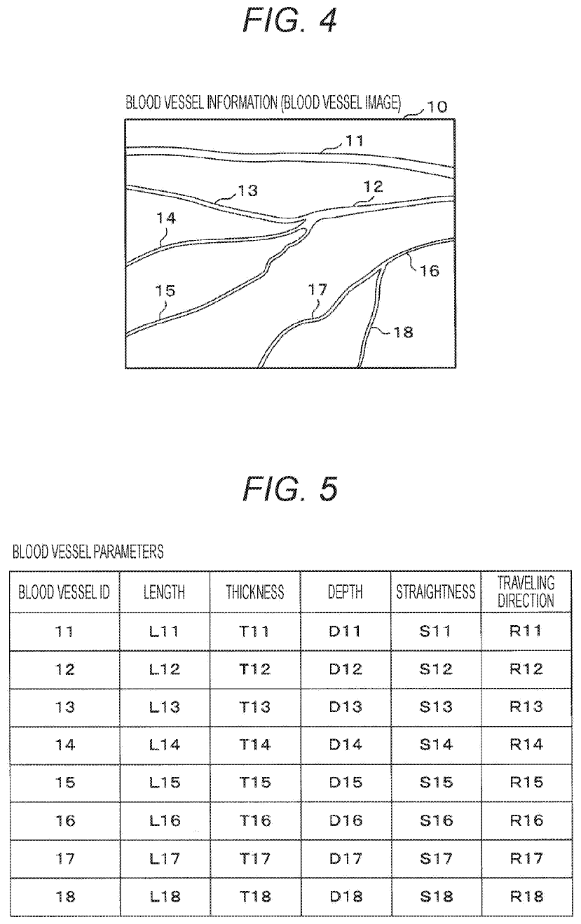

FIG. 4 is a diagram illustrating an example of blood vessel information (blood vessel image). The control unit 51 emits near-infrared light from the light source unit 53 toward the arm of the subject, and acquires an image obtained by imaging the arm irradiated with the near-infrared light with the camera unit 54. Hemoglobin contained in blood has a property of absorbing near-infrared light, and when this property is used, in the obtained image, a portion corresponding to a blood vessel appears black, and a portion other than the blood vessel appears brighter than the portion corresponding to the blood vessel.

The image processing unit 58 can obtain blood vessel information (blood vessel image) by performing edge detection processing or binarization processing on the obtained image to distinguish between the blood vessel and a portion other than the blood vessel. In the example in FIG. 4, blood vessel information (blood vessel image) 10 includes blood vessels 11, 12, 13, 14, 15, 16, 17, and 18.

As described above, the control unit 51 can detect the blood vessel information of the blood vessel included in the predetermined site of the subject on the basis of the image obtained by imaging the predetermined site.

Detection of blood vessel information is not limited to edge detection or binarization processing in the image processing unit 58. For example, a learning model (one learning model of the learning model unit 61) trained to output a blood vessel shape in a case where an image obtained by imaging a predetermined site of a subject is input may be used. In addition, a thermal camera that emits far-infrared light from the light source unit 53 and detects the temperature of the skin surface irradiated with the far-infrared light by the camera unit 54 may be used in combination.

Next, a method of calculating blood vessel parameters will be described.

FIG. 5 is a diagram illustrating an example of blood vessel parameters. The blood vessel parameters may be used to select a blood vessel to be punctured (for example, selection of a puncture optimum blood vessel and determination of priority of a blood vessel to be punctured), and may be used to select a projection region of the virtual operation unit while avoiding a blood vessel region included in a projection image of a blood vessel. The blood vessel parameters include at least one of a length, a thickness, a depth, straightness, and a traveling method of a blood vessel.

The length of the blood vessel is a distance from one end to the other end of the blood vessel. In general, a longer straight blood vessel is easier to puncture. The thickness of the blood vessel is a distance in a direction orthogonal to the length direction of the blood vessel (width of the blood vessel). In general, a larger blood vessel is easier to puncture. The depth of the blood vessel is a distance from the skin surface to the blood vessel. When light is emitted from the light source unit 53, the depth of the blood vessel may be calculated by measuring an ultrasonic wave generated in the blood vessel that has selectively absorbed light energy. In general, a blood vessel located at a position shallower from the skin is easier to puncture. The straightness of a blood vessel may be calculated by thinning a blood vessel image to generate an approximate line and using a determination coefficient (R2) indicating how much the generated approximate line applies to a straight line. The closer the determination coefficient is to 1, the higher the straightness is, and the easier the puncture is. The traveling direction of the blood vessel is an angle between the direction of the approximate line of the blood vessel and a specific direction. The specific direction may be, for example, a direction of the puncture needle when the puncture operator performs puncturing. The puncture needle includes both an inner needle and an outer needle. The inner needle is made of metal and has a role for insertion into a blood vessel. In the outer needle, for example, only the outer needle is indwelled in the blood vessel by puncturing the blood vessel with the inner needle by using a plastic soft catheter and then removing the inner needle.

In the example in FIG. 5, regarding the blood vessel 11, a length, a thickness, a depth, a straightness, and a traveling method of the blood vessel are represented by L11, T11, D11, S11, and R11 for convenience. The same applies to the other blood vessels 12 to 18.

As described above, the control unit 51 may calculate the blood vessel parameters including at least one of the length, the thickness, the depth, the straightness, and the traveling direction of the blood vessel on the basis of the detected blood vessel information.

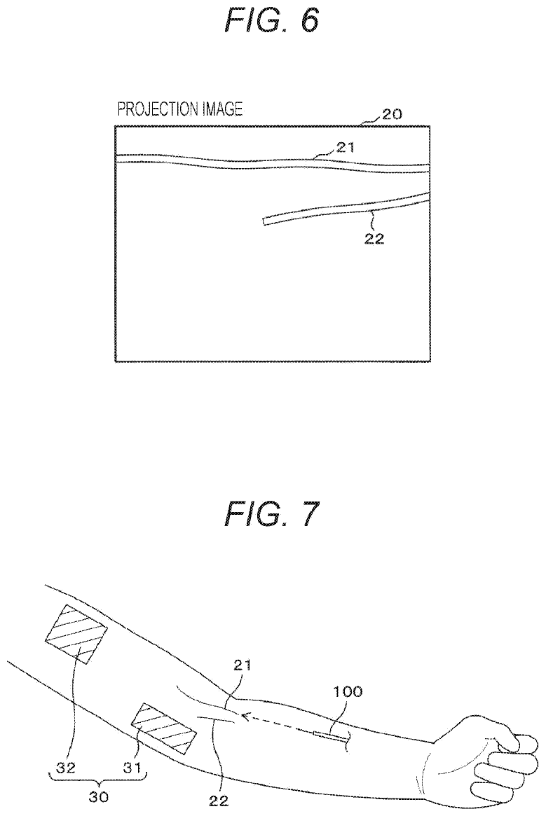

FIG. 6 is a diagram illustrating an example of the projection image 20 of a blood vessel. It is possible to select a blood vessel suitable for puncture by comparing values of the blood vessel parameters (the length, the thickness, the depth, the straightness, and the traveling method of the blood vessel) of the respective blood vessels 11 to 18 illustrated in FIG. 4 with respective blood vessel parameter thresholds. In the example in FIG. 6, the blood vessels 11 and 12 are selected from among the blood vessels 11 to 18, and the projection image 20 is generated on the basis of the blood vessel information of the blood vessels 11 and 12. The blood vessel regions 21 and 22 in the projection image 20 correspond to the blood vessels 11 and 12 in the blood vessel information 10. The blood vessel regions 21 and 22 may be generated by simplifying the shapes of the actual blood vessels 11 and 12. For example, the outer diameter shape of the blood vessel may be alternatively displayed as a straight line or the like inside the blood vessel within a range not exceeding the outer diameter shape.

As described above, the control unit 51 may generate the projection image of the blood vessel on the basis of the detected blood vessel information.

Next, projection of the virtual operation unit will be described.

First, the control unit 51 may detect a projectable region of the virtual operation unit and project the virtual operation unit onto the detected projectable region excluding the blood vessel region of the projection image. The virtual operation unit is located in the region excluding the blood vessel region, and thus the virtual operation unit does not interfere with puncture. As the projectable region of the virtual operation unit, for example, the following method may be used.

(1) Image processing such as edge detection is performed on an image captured by the camera unit 54 to detect a projectable region. In this case, light may be emitted from the light source unit 53, or light may be emitted from the projection unit 55.

(2) The far-infrared light may be emitted from the light source unit 53, and the far-infrared light may be imaged (in combination with a thermal camera) by the camera unit 54.

(3) In addition, as a method of detecting a moving object, a method using a learning model (one learning model in the learning model unit 61) trained to output a projectable region in a case where an image of the arm or the periphery thereof is input may be used. A method may be used in which markers for defining the projectable region are attached to a plurality of necessary locations of the arm, and a region surrounded by the markers is detected to obtain the projectable region.

FIG. 7 is a diagram illustrating an example of a projection region 30 of the virtual operation unit. In FIG. 7, a projection image including blood vessel regions 21 and 22 is projected onto the skin of the arm and displayed. The puncture operator may puncture the blood vessel with the puncture needle 100 with reference to the blood vessel regions of the projection image. In the example in FIG. 7, the blood vessel region 21 is selected as a puncture blood vessel.

When projecting the generated projection image, the control unit 51 may project a virtual operation unit for operating the projection image onto a region (projection region 30) excluding the blood vessel regions 21 and 22 included in the projection image. In the example in FIG. 7, the projection region 30 of the virtual operation unit is divided into two projection regions 31 and 32 and projected. The projection region may be one or more. The virtual operation unit may be projected in the blood vessel region. For example, in a case where a specific blood vessel region may be visually checked on an arm of a puncture subject or the like, the puncture operator may select a projection image of a blood vessel to be projected manually or semi-automatically by directly touching the specific blood vessel region desired to be visualized. In addition, infection can be prevented by using the virtual operation unit.

The control unit 51 may specify a projection recommendation region of the virtual operation unit and project the virtual operation unit onto the specified projection recommendation region. The projection recommendation region is a region that does not cover at least a blood vessel region of the projection image. As a result, the virtual operation unit can be projected at an optimum position that does not interfere with puncture.

In addition, the projection recommendation region is a region that does not cover the blood vessel region of the projection image, and is a region where the finger or the hand of the puncture operator does not exist. The control unit 51 may detect a movement range of the arm including the finger of the puncture operator at a predetermined site and specify the projection recommendation region by excluding the detected movement range. As a result, the virtual operation unit can be projected at an optimum position that does not interfere with puncture. In addition, puncture with less discomfort (for example, a projection image is not projected onto the arm including the finger of the puncture operator) can be performed. In addition, the projection recommendation region is a region where the finger or hand of the puncture operator does not exist and is a region closer to the puncture operator (a region closer to the puncture operator). By locating the projection recommendation region closer to the puncture operator and projecting the virtual operation unit, the puncture operator can easily operate the virtual operation unit and can smoothly perform a button operation.

It is possible to select a blood vessel to be punctured on the basis of the blood vessel parameters calculated on the basis of the above-described blood vessel information and a blood vessel that avoids projection of the virtual operation unit.

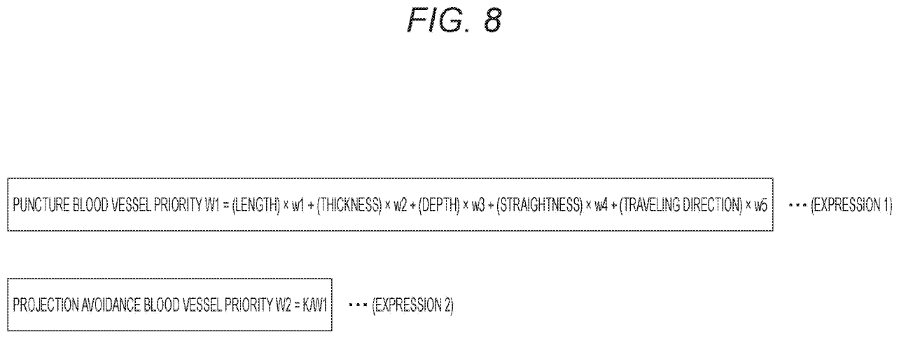

Expression 1 in FIG. 8 illustrates an example of a method of calculating the puncture blood vessel priority and the projection avoidance blood vessel priority. Weighting coefficients for weighting respective values of a length, a thickness, a depth, a straightness, and a traveling method of a blood vessel, which are blood vessel parameters, are indicated by w1, w2, w3, w4, and w5. w1 to w5 may also take 0. The puncture blood vessel priority W1 may be calculated by an expression of W1=(length)×w1+(thickness)×w2+(depth)×w3+(straightness) ×w4+(traveling direction)×w5. The puncture blood vessel priority W1 of a blood vessel may be calculated from the blood vessel information of the blood vessel included in a predetermined portion. The blood vessel having the greatest value of the puncture blood vessel priority W1 may be set as a puncture optimal blood vessel.

The control unit 51 may specify the puncture optimal blood vessel region in the blood vessel region included in the projection image on the basis of the blood vessel parameters, and project the virtual operation unit onto a region excluding the puncture optimal blood vessel region. By displaying a blood vessel suitable for puncture, evaluation of the blood vessel can be performed quickly and accurately.

In addition, a learning model (one learning model of the learning model unit 61) trained to output a puncture success ratio or puncture success/failure in a case where training data including values of the blood vessel parameters and a puncture success ratio or puncture success/failure with respect to a blood vessel having the blood vessel parameters of the values is collected and the values of the blood vessel parameters are input by using the collected training data may be used.

In addition, as illustrated in Expression 2 in FIG. 8, the projection avoidance blood vessel priority W2 may be calculated by an expression of W2=K/W1. K is a constant. The lower (smaller) the puncture blood vessel priority W1, the higher (larger) the projection avoidance blood vessel priority W2.

The control unit 51 may calculate the projection avoidance priority of the blood vessel region included in the projection image by performing weighting with the calculated blood vessel parameters, and select the projection region in which the virtual operation unit is to be projected on the basis of the calculated priority. Through weighting, the virtual operation unit can be projected not to interfere with the blood vessel suitable for puncture even in a case where the projection range is narrow.

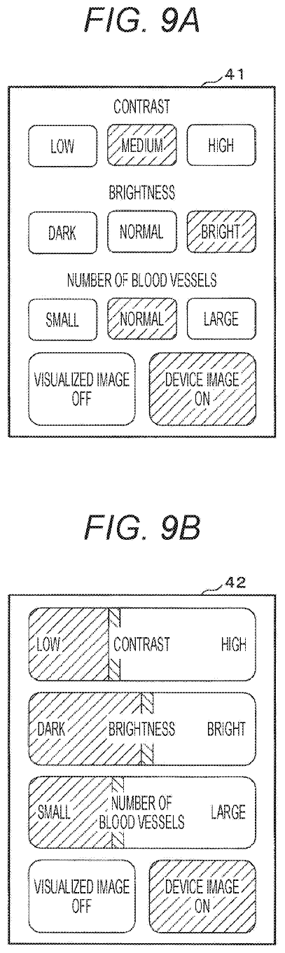

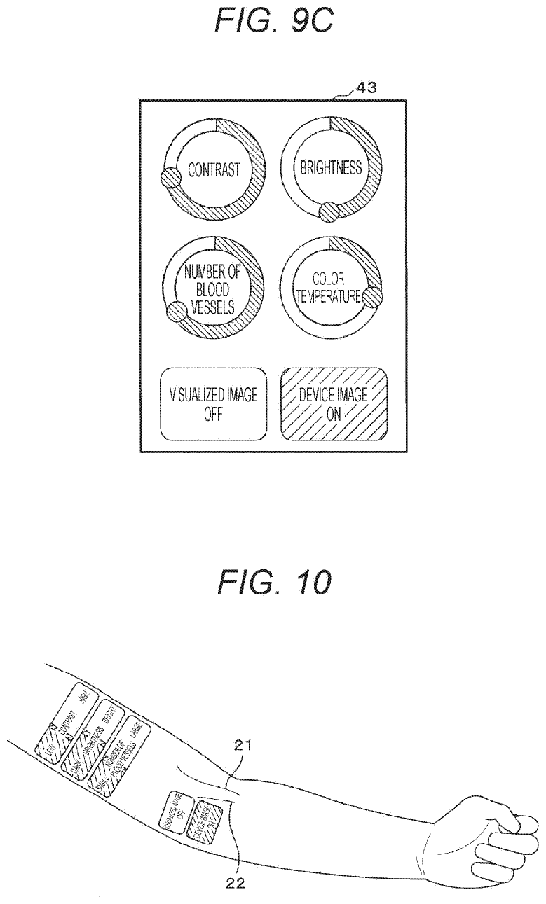

FIGS. 9A to 9C are diagrams illustrating examples of the virtual operation unit. Items that are operable on the virtual operation unit are, for example, contrast and brightness of a projection image of a blood vessel, the number of blood vessels to be displayed, visualized image ON/OFF, device image ON/OFF, and a blood vessel color temperature, but the items that are operable are not limited to the examples in FIG. 9. In FIG. 9A, an operation button of an operation item of the virtual operation unit may be tapped. In FIG. 9B, a slider bar of an operation item of the virtual operation unit may be slid. In FIG. 9C, a ring button of an operation item of the virtual operation unit may be rotated. The visualized image OFF is an operation button for stopping (turning off display of) projection of the projection image. The device image ON is an operation button for turning on display of a distal end position of a puncture needle of a blood vessel access device. The blood vessel access device includes, for example, a product such as a peripheral arteriovenous indwelling needle, a dialysis indwelling needle, a PICC, a midline, or a CV catheter. In addition, in the case of the device image ON, a size, a color, and the like of a region indication (for example, a position thereof is surrounded by a circular mark) for indicating the distal end position of the puncture needle of the blood vessel access device may be changed.

Although not illustrated in FIG. 9, a button (button ON/OFF) for selecting whether to project only the puncture optimal blood vessel or to project all the blood vessels may be projected. In addition, operation buttons for edge adjustment, luminance adjustment, and the like of a blood vessel may be projected.

FIG. 10 is a diagram illustrating an example of a virtual operation unit during projection. In the example in FIG. 10, the projection region is divided into two regions. Of the two projection regions, an item with high priority among the operation items of the virtual operation unit may be disposed in a projection region close to the puncture operator. In the example in FIG. 10, among the operation items of the virtual operation unit, “visualization image OFF” and “device image ON” are projected onto the projection region closer to the puncture operator, and the remaining operation items (contrast, brightness, and the number of blood vessels) of the virtual operation unit are projected onto the projection region farther from the puncture operator.

Note that, in the example in FIG. 10, the virtual operation unit is projected onto the surface of the arm of the subject, but the present invention is not limited thereto. For example, the virtual operation unit may be projected onto a surface of a table or a desk on which the arm of the subject is placed, at a position close to the puncture operator.

The puncture operator may change the display of the projection image according to an operation item by operating the operation item of the virtual operation unit illustrated in FIGS. 9 and 10. For example, when “visualization image OFF” is operated, the projection of the projection image of the blood vessel can be stopped. When “device image ON” is operated, the position of the blood vessel access device can be displayed in the projection image. In addition, when “contrast”, “brightness”, and “number of blood vessels” are operated, the contrast of the projection image, the brightness of the projection image, and the number of blood vessels in the projection image can be changed, respectively.

As described above, the control unit 51 may detect a direct operation on the virtual operation unit and change the display of the projection image according to the detected operation. The direct operation indicates a case where a direct operation is performed on the virtual operation unit.

Furthermore, the control unit 51 may select an operation target object of the virtual operation unit according to the priorities of the plurality of operation target objects included in the virtual operation unit, and project the selected operation target object. Even in a case where the projection range is narrow, the virtual operation unit can be projected while maintaining ease of use.

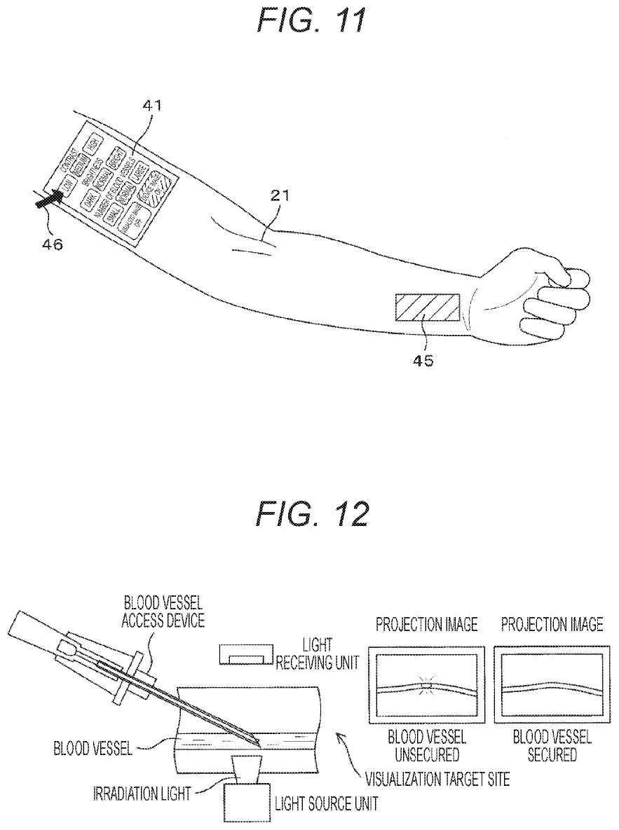

FIG. 11 is a diagram illustrating an example of an indirect operation on the virtual operation unit. In the example in FIG. 11, a virtual operation unit 41 is projected onto the upper arm of the subject. Furthermore, an indirect operation region 45 is projected onto the arm of the subject. When the puncture operator slides the finger on the indirect operation region 45, a pointer 46 on the virtual operation unit 41 moves in conjunction with the sliding of the finger, and a desired operation item can be selected. The puncture operator may indirectly operate the operation item of the virtual operation unit 41 by tapping the finger on the indirect operation region 45. Note that, in the example in FIG. 11, the indirect operation region 45 is projected onto the surface of the arm of the subject, but the present invention is not limited thereto. For example, the indirect operation region 45 may be projected onto a surface of a table or a desk on which the arm of the subject is placed, at a position close to the puncture operator.

As described above, the control unit 51 can detect an indirect operation on the virtual operation unit and change the display of the projection image according to the detected operation. The indirect operation indicates a case where the virtual operation unit is indirectly operated via an operation unit different from the virtual operation unit. By changing the display of the projection image, the virtual operation unit can be projected in a state of being optimal for puncture.

FIG. 12 is a diagram illustrating an example of a configuration of a blood vessel visualization system including a blood vessel access device. A light source unit that emits near-infrared light and a light receiving unit are disposed with a visualization target site interposed therebetween. Near-infrared light is emitted from the light source unit, light transmitted through the visualization target site is received by the light receiving unit, a projection image of a blood vessel is generated and projected, a position of a distal end of a puncture needle of the blood vessel access device is detected, and the detected position of the distal end of the puncture needle is displayed on a projection image. In this case, when a light emitting portion of the distal end of the puncture needle reaches the blood vessel, the light of the light emitting portion disappears, and it is possible to visually recognize that the puncture needle secures the blood vessel.

The blood vessel access device includes, for example, a peripheral arteriovenous indwelling needle, a dialysis indwelling needle, a PICC, a midline, or a CV catheter, and a distal end of a catheter or an inner needle distal portion emits, fluoresces, or reflects near-infrared light.

Note that the light source unit and the camera unit 54 may be disposed on the same side with respect to the visualization target site, and reflected light of near-infrared light emitted from the light source unit may be detected. Furthermore, the camera unit 54 may be provided in addition to the light receiving unit.

In the above-described blood vessel visualization system, the control unit 51 can detect a position of the blood vessel access device that punctures a predetermined site on the basis of an image obtained by imaging the predetermined site, and project the virtual operation unit onto a region other than the detected position. By detecting the position of the blood vessel access device, the virtual operation unit may be projected without interfering with puncture.

In addition, by combining the blood vessel visualization device 50 of the present embodiment and a semi-automatic puncture robot, a puncture blood vessel is evaluated with the assistance of the blood vessel visualization device 50, and a medical worker makes the final decision of the puncture, so that the button operation can be quickly performed, and it is expected that the puncture is smoothly performed.

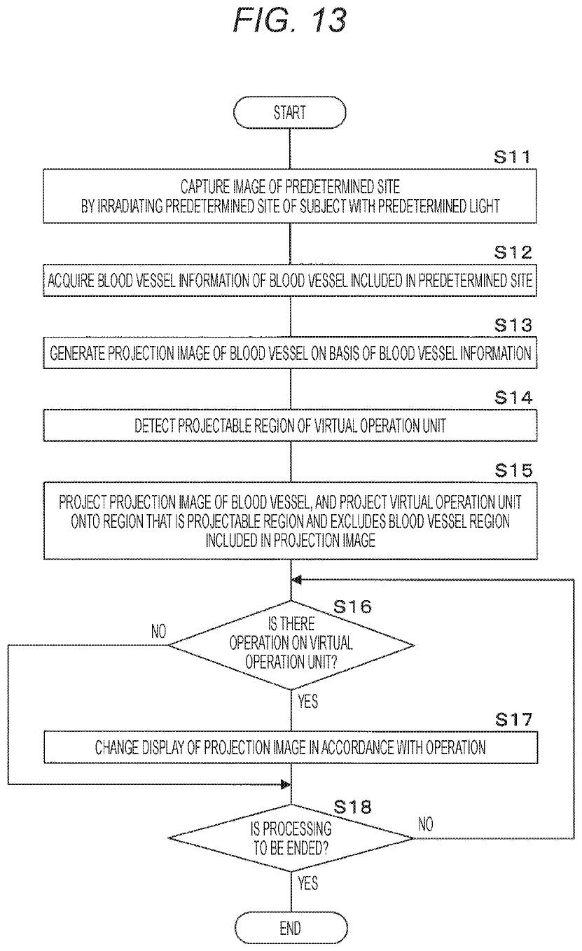

FIG. 13 is a diagram illustrating an example of a processing procedure executed by the blood vessel visualization device 50. Hereinafter, for convenience, the control unit 51 will be described as a subject of the processing. The control unit 51 irradiates a predetermined site of a subject with predetermined light to image the predetermined site (S11). The predetermined light is, for example, near-infrared light. The predetermined light may include visible light and far-infrared light. The control unit 51 acquires blood vessel information of a blood vessel included in the predetermined site (S12), and generates a projection image of the blood vessel on the basis of the acquired blood vessel information (S13). See FIG. 4 for blood vessel information. See FIG. 6 for projection images.

The control unit 51 detects a projectable region of the virtual operation unit (S14), projects the projection image of the blood vessel, and projects the virtual operation unit onto a region that is the projectable region and excludes the blood vessel region included in the projection image (S15). In a case where the puncture optimal blood vessel is selected, the virtual operation unit may be projected onto a blood vessel region as long as the blood vessel region is not the puncture optimal blood vessel region. In addition, when the priority of the puncture blood vessel is calculated and a plurality of puncture candidate blood vessels are selected according to the priority, the virtual operation unit may be projected onto a blood vessel region as long as the blood vessel region is not the selected puncture candidate blood vessel regions.

The control unit 51 determines whether or not there is an operation (a direct operation or an indirect operation) on the virtual operation unit (S16), and if there is an operation (YES in S16), changes the display of the projection image according to the operation (S17), and determines whether or not to end the processing (S18). In a case where there is no operation on the virtual operation unit (NO in S16), the control unit 51 performs the processing in step S18. In a case where the processing is not ended (NO in S18), the control unit 51 continues the processing in and after step S16, and in a case where the processing is ended (YES in S18), the processing is ended.

According to the present embodiment, since the virtual operation unit (operation buttons, icons, bars, and the like displayed through projection) that can be operated can be disposed at an appropriate position (for example, the surface may be a skin surface of a predetermined site, or a surface of a stand or a table, or the like on which the predetermined site is placed) that is close to the puncture site and does not interfere with puncture, it is not necessary to operate a device button or the like at a position away from the puncture site, and an actual device button is not required. Therefore, there is no concern about infection via the device button. Accordingly, it is possible to support easy puncturing without operating the device button.

### REFERENCE CHARACTER LIST

10 Blood vessel information
11, 12, 13, 14, 15, 16, 17, 18 Blood vessel
20 Projection image
21, 22 Blood vessel region
50 Blood vessel visualization device
51 Control unit
52 Communication unit
53 Light source unit
54 Camera unit
55 Projection unit
56 Sensor unit
57 Memory
58 Image processing unit
59 Storage unit
60 Computer program
61 Learning model unit
100 Puncture needle

1. A non-transitory computer-readable medium containing a computer program that, when executed, causes a computer to execute processing comprising:
detecting blood vessel information of a blood vessel included in a predetermined site of a subject based on an image obtained by imaging the predetermined site;
generating a projection image of the blood vessel based on the detected blood vessel information; and
while causing at least one projector to project the generated projection image, causing the at least one projector to project a virtual operation unit for operating the projection image onto a region excluding a blood vessel region included in the projection image.

2. The non-transitory computer-readable medium according to claim 1, wherein the computer program, when executed, causes the computer to execute processing comprising:
detecting a projectable region of the virtual operation unit; and
causing the at least one projector to project the virtual operation unit onto a region that is the detected projectable region and excludes the blood vessel region.

3. The non-transitory computer-readable medium according to claim 1, wherein the computer program, when executed, causes the computer to execute processing comprising:
detecting an operation on the virtual operation unit; and
changing display of the projection image in accordance with the detected operation.

4. The non-transitory computer-readable medium according to claim 1, wherein the computer program, when executed, causes the computer to execute processing comprising:
specifying a projection recommendation region of the virtual operation unit; and
causing the at least one projector to project the virtual operation unit onto the specified projection recommendation region.

5. The non-transitory computer-readable medium according to claim 4, wherein the computer program, when executed, causes the computer to execute processing comprising:
detecting a movement range of an arm including a finger of a puncture operator at the predetermined site; and
specifying the projection recommendation region by excluding the detected movement range.

6. The non-transitory computer-readable medium according to claim 5, wherein:
the projection recommendation region is a region excluding the movement range and includes a region close to the puncture operator.

7. The non-transitory computer-readable medium according to claim 1, wherein the computer program, when executed, causes the computer to execute processing comprising:
calculating blood vessel parameters including at least one of a length, a thickness, a depth, a straightness, and a traveling direction of the blood vessel based on the detected blood vessel information; and
specifying a puncture optimal blood vessel region among blood vessel regions included in the projection image based on the calculated blood vessel parameters; and
causing the at least one projector to project the virtual operation unit onto a region excluding the puncture optimal blood vessel region.

8. The non-transitory computer-readable medium according to claim 1, wherein the computer program, when executed, causes the computer to execute processing comprising:
calculating blood vessel parameters including at least one of a length, a thickness, a depth, a straightness, and a traveling direction of the blood vessel based on detected blood vessel information;
calculating a projection avoidance priority of a blood vessel region included in the projection image by performing weighting with the calculated blood vessel parameters; and
selecting a projection region onto which the virtual operation unit is to be projected based on the calculated priority.

9. The non-transitory computer-readable medium according to claim 1, wherein the computer program, when executed, causes the computer to execute processing comprising:
detecting a position of a blood vessel access device that punctures the predetermined site based on the image obtained by imaging the predetermined site; and
causing the at least one projector to project the virtual operation unit onto a region other than the detected position.

10. The non-transitory computer-readable medium according to claim 1, wherein the computer program, when executed, causes the computer to execute processing comprising:
selecting an operation target object of the virtual operation unit according to priorities of a plurality of operation target objects included in the virtual operation unit; and
causing the one or more projectors to project the selected operation target object.

11. A blood vessel visualization device comprising:
a control unit configured to:
detect blood vessel information of a blood vessel included in a predetermined site of a subject based on an image obtained by imaging the predetermined site,
generate a projection image of the blood vessel based on the detected blood vessel information, and
while causing one or more projectors to project the generated projection image, cause the one or more projectors to project a virtual operation unit for operating the projection image onto a region excluding a blood vessel region included in the projection image.

12. A blood vessel visualization system comprising:
the blood vessel visualization device according to claim 11; and
a blood vessel access device configured to puncture the predetermined site of the subject.

13. The blood vessel visualization system according to claim 11, wherein:
the control unit is configured to:
detect a projectable region of the virtual operation unit, and
cause the at least one projector to project the virtual operation unit onto a region that is the detected projectable region and excludes the blood vessel region.

14. The blood vessel visualization system according to claim 11, wherein:
the control unit is configured to:
detect an operation on the virtual operation unit; and
change display of the projection image in accordance with the detected operation.

15. The blood vessel visualization system according to claim 11, wherein:
the control unit is configured to:
specify a projection recommendation region of the virtual operation unit, and
cause the at least one projector to project the virtual operation unit onto the specified projection recommendation region.

16. The blood vessel visualization system according to claim 15, wherein:
the control unit is configured to:
detect a movement range of an arm including a finger of a puncture operator at the predetermined site, and
specify the projection recommendation region by excluding the detected movement range.

17. The blood vessel visualization system according to claim 15, wherein:
the projection recommendation region is a region excluding the movement range and includes a region close to the puncture operator.

18. The blood vessel visualization system according to claim 11, wherein:
the control unit is configured to:
calculate blood vessel parameters including at least one of a length, a thickness, a depth, a straightness, and a traveling direction of the blood vessel based on the detected blood vessel information,
specify a puncture optimal blood vessel region among blood vessel regions included in the projection image based on the calculated blood vessel parameters, and
cause the one or more projectors to project the virtual operation unit onto a region excluding the puncture optimal blood vessel region.

19. The blood vessel visualization system according to claim 11, wherein:
the control unit is configured to:
calculate blood vessel parameters including at least one of a length, a thickness, a depth, a straightness, and a traveling direction of the blood vessel based on detected blood vessel information,
calculate a projection avoidance priority of a blood vessel region included in the projection image by performing weighting with the calculated blood vessel parameters, and
select a projection region onto which the virtual operation unit is to be projected based on the calculated priority.

20. A blood vessel visualization method comprising:
detecting blood vessel information of a blood vessel included in a predetermined site of a subject based on an image obtained by imaging the predetermined site;
generating a projection image of the blood vessel based on the detected blood vessel information; and
while projecting the generated projection image, projecting a virtual operation unit for operating the projection image onto a region excluding a blood vessel region included in the projection image.
